(19) United States
(12) Patent Application Publication
(10) Pub. No.: US 2024/0336911 A1
(43) Pub. Date: Oct. 10, 2024

(54) MICROFLUIDIC DEVICE FOR EXTRACTING DNA

(71) Applicant: Oujiang Laboratory, Wenzhou City (CN)

(72) Inventors: Nasir JALAL, Wenzhou City (CN); Peter Edward SHAW, Wenzhou City (CN); He LIU, Wenzhou City (CN)

(21) Appl. No.: 18/361,006

(22) Filed: Jul. 28, 2023

(30) Foreign Application Priority Data
Apr. 10, 2023 (CN) 2023104104761

(51) Int. Cl. C12N 15/10; C12Q 1/6806

(52) U.S. Cl. CPC C12N 15/1017; C12Q 1/6806

(57) ABSTRACT

A microfluidic device for extracting DNA is provided. The microfluidic device includes a feeder layer, a buffer layer and a pneumatic layer which are sealed and stacked in sequence. An electromagnetic plate is arranged between the buffer layer and the pneumatic layer. The feeder layer is provided with multiple main channels and three blister openings. A syringe pump is connected to an inlet of each main channel. The buffer layer is provided with segmented buffer channels, each segmented buffer channel is provided with multiple subchannels, and each segmented buffer channel corresponds to one pop-type blister. The buffer channel is communicated with a main channel. The pop-type blister is communicated with an inlet of the segmented buffer channel. The pop-type blister is arranged at the top of the feeder layer and is communicated with the inlet of the segmented buffer channel through the blister opening.

### CROSS-REFERENCE TO RELATED APPLICATION

This patent application claims the benefit and priority of Chinese Patent Application No. 2023104104761 filed with the China National Intellectual Property Administration on Apr. 10, 2023, the disclosure of which is incorporated by reference herein in its entirety as part of the present application.

### TECHNICAL FIELD

The present disclosure relates to the field of DNA extraction, and in particular to a microfluidic device for extracting DNA.

### BACKGROUND

Friedrich Miescher first extracted DNA from human white blood cells in 1869. Nowadays, DNA extracted by many diagnostic and research laboratories usually requires extraction from various cell types, such as bacteria, fungi, plasmodium, plants, animals, and humans.

DNA extraction is usually a first step in the following related molecular studies: 1. Disease diagnosis; 2. Mutation detection and cancer gene analysis; 3. Identification and evidence collection of genetic biomarkers; 4. Genetic engineering and cloning; 5. Paternity testing/Lineage tracing. 6. Sex determination of many ornamental birds such as a love bird; 7. DNA sequencing (Sanger and next-generation sequencing).

When DNA is extracted from bacteria, fungi, plants, animals, or humans, multiple samples are usually run simultaneously. Therefore, the DNA extraction process must be cost-effective, rapid and should provide high-quality DNA in large quantities. Current methods for DNA extraction can be divided into three categories according to use of separation agents (i.e., chemical, centrifugal or magnetic): classical (manual) chemical methods; modern (semi-automatic/centrifugal) centrifugal column methods; and modern (fully automatic) magnetic methods.

According to user-friendliness and several DNA samples that can run in parallel at any given time, the DNA extraction methods can be divided into low-throughput methods or high-throughput methods. With the increasing burden of processing population samples in diagnostic laboratories and the need to analyze DNA for various purposes, it is almost inevitable to adopt a timesaving, high-throughput, and low-cost method. The current methods are either low cost or high throughput, but the low cost and the high throughput methods have not yet been combined.

There are several marketed kits that allow users to efficiently extract DNA from sample cells using principles of lysis, binding, washing, and elution (Carpi F M, Di Pietro F, Vincenzetti S, Mignini F, Napolioni V. Human DNA extraction methods: patents and applications. Recent Pat DNA Gene Seq. 2011; 5 (1): 1-7); Tan S C, Yiap B C. DNA, RNA, and protein extraction: the past and the present. J Biomed Biotechnol. 2009; 2009:574398).

A set of lysis, binding, washing and elution buffer solutions are used in most DNA extraction methods, these buffer solutions have been used by many researchers (Shi R, Lewis R S, Panthee D R (2018) Filter paper-based spin column method for cost-effective DNA or RNA purification. PLOS ONE 13 (12): e0203011. Https://doi.org/10.1371/journal.pone.0203011). or magnetic beads are used in combination with these buffer solutions.

In the fastest automated DNA extraction method at present, magnetic beads are released into the whole cell lysate to start the DNA capture and binding process. DNA extraction with magnetic beads began in 1990s, which can be proved by the patent “DNA purification and separation with magnetic beads” authorized by the United States. The method remains basically unchanged, relying on the use of commercially available (https://www.cytivalifesciences.com/en/us/news-center/magnetic-beads-a-simple-guide-10001) coated magnetic beads (20 μM carboxylate modified magnetite beads), and the magnetic beads can be reversibly bound to nucleic acid by adjusting buffer solution conditions. After the DNA is bound, the beads are attracted by an external magnetic field to an outer edge of the channel and immobilized. When the magnetic beads are immobilized, the DNA bound to the magnetic beads remains in the washing step. Lastly, the elution buffer solution is added and the magnetic field is removed, and then the DNA is released as a purified sample for quantification and analysis.

Compared with the main DNA extraction methods currently used by researchers or diagnostic laboratories around the world, Table 1 is a parameter table of different DNA extraction methods provided by the present disclosure, FIGS. 1A-1C is a schematic diagram of different DNA extraction methods, FIG. 1A is a schematic diagram of DNA extraction based on a phenol chloroform (organic) method, which usually takes one to two days, FIG. 1B is a schematic diagram of DNA extraction based on a silica gel spin column (semi-automatic) method, which usually takes one to two hours, and FIG. 1C is a schematic diagram of DNA extraction based on a magnetic bead (automatic) method, which usually takes less than 20 minutes, as shown in Table 1 and FIGS. 1A-1C.

TABLE 1

| | | Toxic | Cost/ | | Number of |
|---|---|---|---|---|---|
| | Raw | compound/ | sample | Required | samples |
| Method | materials | solvent | (USD) | Time | per run |
| Phenol | Whole | Phenol, | <5 | >1-2 | 1 |
| chloroform | blood, | chloroform | | days | |
| | cultured | | | | |
| | cells, tissues | | | | |
| Silica | Whole | Phenol, | <5 | 1-2 | 1 |
| gel | blood, | chloroform, | | hours | |
| | cultured | or guanidine | | | |
| | cells, tissues | thiocyanate | | | |
| Benzyl | Whole | Benzyl | <5 | >3 | 1 |
| alcohol | blood, | alcohol | | hours | |
| | cultured | | | | |
| | cells, tissues | | | | |
| Salting | Whole | None | <5 | >3 | 1 |
| out | blood, | | | hours | |
| | cultured | | | | |
| | cells, tissues | | | | |
| Extracting | Buccal | Formamide | <5 | 2 | 1 |
| from dry | smear, | | | hours | |
| specimens | semen, and | | | | |
| | dried blood | | | | |
| Chromatography | Any | None | <5 | <30 | 1 |
| | sample | | | minutes | |
| Magnetic | Whole | None | >5 | <30 | Multiple |
| bead | blood, | | | minutes | |
| | cultured | | | | |
| | cells, tissues | | | | |
| Dendritic | Whole | None | >10 | <30 | |
| polymer | blood, | | | minutes | |
| | cultured | | | | |
| | cells, tissues | | | | |
| Matrix | Small | None | >30 | <30 | Multiple |
| mill | samples | | | minutes | |
| | from organic | | | | |
| | sources | | | | |
| Mitochondrial | Hair | Phenol, | >5 | >24 | 1 |
| DNA | | chloroform | | hours | |
| extraction | | | | | |
| Mitochondrial | Hair, teeth | Guanidinium | >5 | >24 | 1 |
| DNA | | Thiocyanate | | hours | |
| extraction | | | | | |
| Mitochondrial | Hair, teeth, | None | >5 | 2 | 1 |
| DNA | skeleton | | | hours | |
| extraction | | | | | |
| Miniature | Cell | None | >20 | <30 | 1 |
| apparatus | suspension | | | minutes | |
| Rapid μFDA | Whole | Guanidinium | <5 | <10 | 8 |
| | blood, | Thiocyanate | | minutes | |
| | cultured | | | | |
| | cells, plants, | | | | |
| | insects, | | | | |
| | tissues, | | | | |
| | bacteria, | | | | |
| | fungi | | | | |

It can be seen from the above that DNA extraction based on the magnetic bead method takes the shortest time, but most high-throughput methods take advantage of the flexibility of magnetic bead determination, and expensive robots need to be installed for the use of a mechanized DNA extraction platform, leading to extremely high costs per sample.

The way to reduce the cost for each test is to sign a long-term contract with the end user and provide robots free of charge. As long as the end user continues to use the same closed platform robot and purchases DNA extraction tests from the same manufacturer, the price of each sample may be 5 to 10 dollars. Some open platforms have improved mechanisms to allow tests from any manufacturer to run on the robot, but even so, cost remains an issue. In addition, these robot platforms are not feasible for small and medium-sized users such as academic researchers, because they sometimes use ten to hundreds of samples every day, but cannot afford to install commercial-scale robots.

### SUMMARY

An objective of some embodiments of the present disclosure is to provide a microfluidic device for extracting DNA, at low cost, ensuring low time consumption yet providing a high throughput method.

To achieve the above objective, the present disclosure provides the following technical solution.

A microfluidic device for extracting DNA includes a feeder layer, a buffer layer and a pneumatic layer which are sealed and stacked in sequence. An electromagnetic plate is arranged between the buffer layer and the pneumatic layer.

The feeder layer is provided with multiple main channels and three blister openings. A syringe pump is connected to an inlet of each main channel, and the syringe pump is configured to inject a lysate sample into each main channel.

The buffer layer is provided with segmented buffer channels. Each segmented buffer channel is provided with multiple subchannels, and each segmented buffer channel corresponds to one pop-up blister. The buffer channel is communicated with the main channel. The pop-up blister is connected to an inlet of each segmented buffer channel. The pop-up blister is arranged at the top of the feeder layer and is communicated with the inlet of each segmented buffer channel through the blister opening. Each pop-up blister is filled with different type of buffer solutions, and these buffer solutions include a magnetic binding buffer solution, a washing buffer solution, and an elution buffer solution.

Each segmented buffer channel is provided with multiple convergence nodes, a pop-up valve is arranged at each convergence node, and the pop-type valve is pneumatically controlled by the pneumatic layer.

Alternatively, the feeder layer, the buffer layer, and the pneumatic layer, each have a length of 10 cm and a thickness of 1 mm.

Alternatively, a collection device is arranged at an outlet of each main channel. The collection device is configured to collect a DNA sample.

Alternatively, a pop-up blister containing the magnetic binding buffer solution, a pop-up blister containing the washing buffer solution, and a pop-up blister containing the elution buffer solution are arranged in sequence in a direction from the inlet of the main channel to the outlet of the main channel.

The pop-up blister containing the magnetic binding buffer solution, the pop-up blister containing the washing buffer solution and the pop-up blister containing the elution buffer solution are opened in sequence in the direction from the inlet of the main channel to the outlet of the main channel to release the magnetic binding buffer solution, the washing buffer solution, and the elution buffer solution in sequence.

Alternatively, eight main channels are provided, and each segmented buffer channel includes eight subchannels.

Alternatively, each segmented buffer channel is provided with an eight-valve channel separator with three pop-up valves.

Alternatively, the pop-up valve is a check valve for limiting fluid from entering one subchannel or eight subchannels simultaneously.

Alternatively, the pop-up valve is a T-shaped free-moving polydimethylsiloxane structure.

Alternatively, the microfluidic device is reusable after washing with a bleaching agent with a concentration of 1% to 2%, and rinsing with double distilled water followed by air-drying.

According to the specific embodiments provided by the present disclosure, the following technical effects are disclosed in the present disclosure. A microfluidic device for extracting DNA is provided, which includes a feeder layer, a buffer layer and a pneumatic layer sealed and stacked in sequence. Based on a magnetic bead method, high-quality and high-quantity DNA is collected simultaneously through multiple channels. Due to the arrangement of an electromagnetic plate and a magnetic binding buffer solution, the time for DNA extraction is further reduced based on the magnetic bead method, and high-throughput pure DNA can be extracted in a short time without the assistance of an expensive robot. Therefore, the extraction cost and time is significantly reduced while combining high yield with a high throughput method.

### BRIEF DESCRIPTION OF THE DRAWINGS

To describe the technical solutions in the embodiments of the present disclosure or in the prior art more clearly, the accompanying drawings required for describing the embodiments will be briefly described below. Apparently, the accompanying drawings described below show merely some embodiments of the present disclosure, and other drawings can be obtained from these accompanying drawings without creative efforts for those of ordinary skill in the art.

FIGS. 1A-1C are a schematic diagram of different DNA extraction methods; FIG. 1A is a schematic diagram of DNA extraction based on a phenol chloroform (organic) method; FIG. 1B is a schematic diagram of DNA extraction based on a silica gel (semi-automatic) method; FIG. 1C is a schematic diagram of DNA extraction based on a magnetic bead (automatic) method.

FIG. 2 is a schematic diagram of an exploded structure of a microfluidic device for extracting DNA in accordance with the present disclosure.

FIG. 3 is a flowchart of a DNA extraction method based on a microfluidic device for extracting DNA in accordance with the present disclosure.

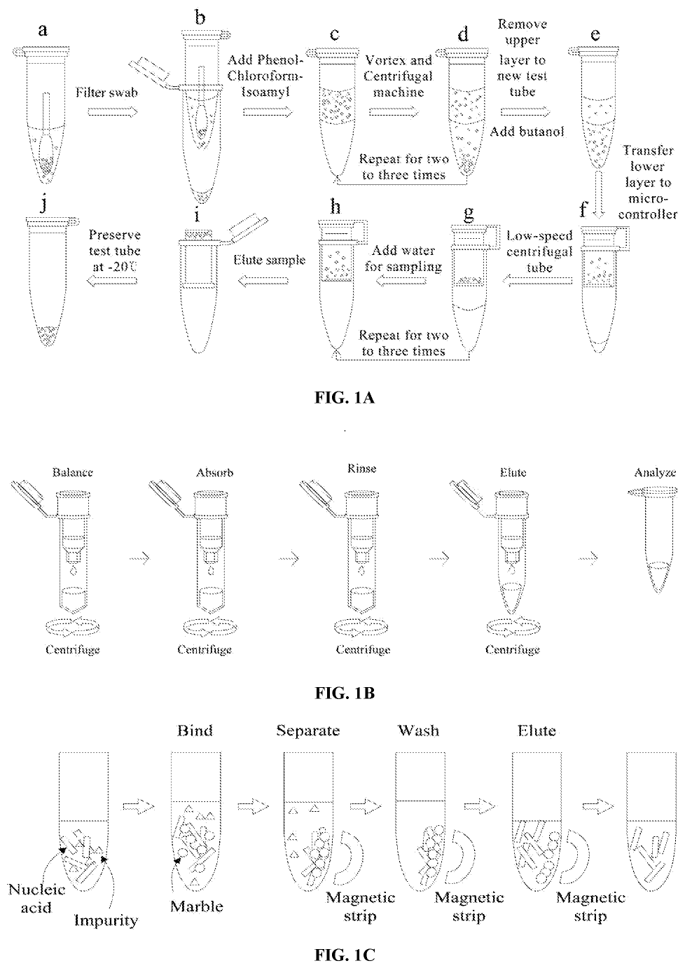

### DETAILED DESCRIPTION OF THE EMBODIMENTS

The technical solutions in the embodiments of the present disclosure are clearly and completely described below with reference to the accompanying drawings in the embodiments of the present disclosure. Apparently, the described embodiments are merely a part rather than all of the embodiments of the present disclosure. All other embodiments obtained by those of ordinary skills in the art based on the embodiments of the present disclosure without creative efforts shall fall within the protection scope of the present disclosure.

An objective of some embodiments of the present disclosure is to provide a microfluidic device for extracting DNA, which can reduce the extraction cost while ensuring low time consumption and high throughput.

To make the objectives, features and advantages of the present disclosure more clearly understood, the present disclosure will be described in further detail below with reference to the accompanying drawings and the detailed description.

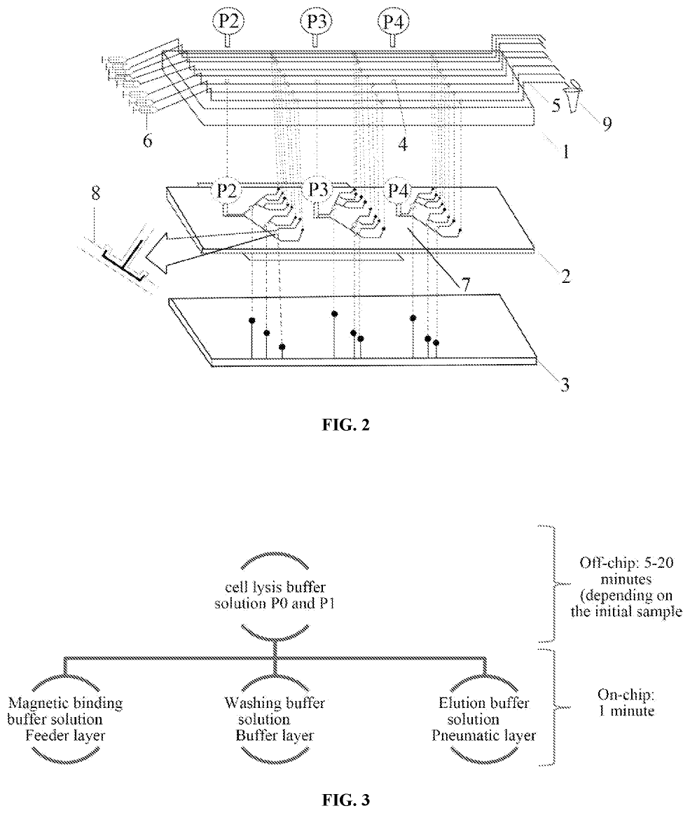

As shown in FIG. 2, the present disclosure provides a microfluidic device (hereinafter referred to as RapidμFDNA for short) for extracting DNA, which includes a feeder layer 1, a buffer layer 2 and a pneumatic layer 3 which are sealed and stacked in sequence. An electromagnetic plate is arranged between the buffer layer and the pneumatic layer. The feeder layer is provided with multiple main channels 5 and three blister openings 4. A syringe pump 6 is connected to an inlet of each main channel 5, and the syringe pump 6 is used to inject a lysate sample to the main channel 5. The buffer layer is provided with segmented buffer channels 7. Each segmented buffer channel 7 is provided with multiple subchannels, and each segmented buffer channel 7 corresponds to one pop-up blister. The buffer channel 7 is communicated with the main channel 5. A pop-up blister is communicated with an inlet of each segmented buffer channel 7. The pop-up blister is arranged at top of the feeder layer and is communicated with the inlet of the buffer channel 7 after passing through the blister opening 4. Each pop-type blister is filled with different types of buffer solution, and the buffer solution includes a magnetic binding buffer solution, a washing buffer solution, and an elution buffer solution. Each segmented buffer channel 7 is provided with multiple convergence nodes. A pop-up valve 8 is arranged at each convergence node, and the pop-up valve 8 is pneumatically controlled by the pneumatic layer.

In practical application, the feeder layer, the buffer layer, and the pneumatic layer all have a length of 10 cm and a thickness of 1 mm.

In practical application, a collection device 9 is arranged at an outlet of each main channel 5. The collection device 9 is used to collect the DNA sample from each main channel 5.

In practical application, a pop-up blister P2 containing the magnetic binding buffer solution, a pop-up blister P3 containing the washing buffer solution and a pop-up blister P4 containing the elution buffer solution are arranged in sequence in a direction from the inlet of the main channel 5 to the outlet of the main channel 5, thus releasing the magnetic binding buffer solution, the washing buffer solution and the elution buffer solution in sequence.

In practical application, eight main channels 5 are provided, and each segmented buffer channel 7 include eight subchannels equipped with intelligently placed valves to direct flow into one to eight channels, that will supply the required buffer solution at specific time to the main channel in the feeder layer.

In practical application, each segmented buffer channel 7 is provided with an eight-valve channel separator with three pop-up valves 8, that allow the user to use one to eight subchannels simultaneously.

In practical application, the pop-up valve 8 is a check valve, and the pop-up valve 8 is used to limit fluid from entering one subchannel of the buffer channel 7 or eight subchannels of the buffer channel 7 simultaneously.

In practical application, the pop-up valve 8 is a T-shaped free-moving polydimethylsiloxane structure, under the control of pneumatic layer.

In practical application, after use, the microfluidic device is washed with a bleaching agent with a concentration of 1% to 2%, is rinsed with double distilled water and then is air-dried.

In practical application, the manufacture of RapidμFDNA mainly includes: based on a microfluidic chip made of polydimethylsiloxane (PDMS), preparing the RapidμFDNA by using standard industrial technologies such as lithography, photomask and photoresist technology used by other companies.

Each layer is made of a polydimethylsiloxane PDMS coating with a thickness of 1 mm. Each subchannel of the buffer layer 2 should be connected to the main channel 5 of the feeder layer 1, while a pneumatic controller of the pneumatic layer 3 is connected to the valves of the buffer layer 2. Therefore, the designed overlapping parts should be perfectly aligned to facilitate the cooperative operation of these channels. Each layer has a length of 10 cm and a thickness of 1 mm, so the total thickness of the three layers is 3 mm, and the length of the three layers is 10 cm.

The three layers are interconnected to one another and work together as one unit. These three layers are sealed together by a process called plasma sealing. The feeder layer 1 is the primary “FEEDER” layer, which delivers the cell lysate to the microfluidic device, while the buffer layer 2 is the “BUFFER” layer, which adds the required buffer solution to the feeder layer 1 at the right time. Finally, the pneumatic layer 3 is a “pneumatic” layer including the pneumatic controller for the valve of the buffer layer 2 and a blister pop-up buffer release control device. These layers are clamped together and sealed to eventually become one unit.

Branch channels in the buffer layer 2 are responsible for directing buffer solutions from a pop-up blister to one, two, three, four, five, six, seven, or all eight subchannels. Once it is determined how many subchannels are required, valves are operated to transfer respective buffer solutions to their subsequent subchannels, and then specific buffer solution is gradually added to the main channel 5 of the feeder layer 1.

Finally, the pneumatic layer 3 is a “pneumatic” layer, including the pneumatic controller for the valve of the buffer layer 2 and blisters. These layers are clamped together and sealed to eventually become one unit. The pneumatic layer 3 controls a pneumatic valve found in the buffer layer 2.

The valve of the buffer layer 2 is controlled by the pneumatic controller of the pneumatic layer 3.

“Overlaying” refers to a “sandwich” design of the microfluidic device, and three layers are connected together.

The main channel in the feeder layer 1 is continuous and has a feeder inlet and outlet. In the buffer layer 2, a specific buffer solution is added to the main channel of the feeder layer 1 at a specific point. The blister contains buffer solution to be released into the subchannel of the buffer layer 2, and the buffer solution is finally brought into the main channel of the feeder layer 1 for mixing.

The blisters (P2, P3, P4) essentially include containers/reservoirs containing buffer solutions (chemicals) required for the DNA extraction process. Buffer solutions from the P2, P3, P4 are released under the control of the pneumatic controller located in the pneumatic layer 3 of the device.

The following three types of liquid are called buffer solutions: a magnetic binding buffer solution contained in P2, a washing buffer solution contained in P3, and an elution buffer solution contained in P4.

The buffer solution is initially released into the subchannel of the buffer layer 2, but gradually enters the main channel of the feeder layer 1 where mixing occurs.

There is only one electromagnet under the pneumatic layer 3, but magnetic force of the electromagnet can act on the buffer layer 2 and the feeder layer 1. The function of the magnetic force is to attract magnetic particles in the magnetic binding buffer solution released from P2. Once the magnetic particles are in contact with the lysate in the feeder layer 1, these magnetic particles bind to DNA, thus the magnet attracts the magnetic particles and DNA. During this process, the magnetic particles separate DNA from the lysate fed into the main channel of the feeder layer 1.

Feeder Layer 1: The layer is the “main layer”, provided with a buffer inlet and includes eight channels. The syringe pump 6 (containing lysate samples 1-8) feeds the lysate into the microfluidic device.

Once the lysate flows into the channel, three bubbles of the buffer layer 2 pop up to push the binding, washing and elution buffer solutions into the channel in sequence. At the end of processing, pure DNA can be collected from a collection port. The electromagnet in the buffer layer 2 plays a role of separating DNA from original lysis solution.

Buffer Layer 2: A buffer solution inlet is a pop-up blister containing 50 μL of magnetic binding buffer solution, in which fine magnetic beads are included. Once the lysate sample (one to eight) is loaded onto the chip and pushed into a feeder channel by a positive pressure controller, a pop-up blister squasher automatically releases the magnetic beads. Such a mechanism described here allows a user to extract DNA from eight independent samples by using one channel or eight channels simultaneously.

In the present disclosure, an eight-pin channel separator with three valves is provided to allow the user to select the number of channels required for DNA extraction. These valves are a T-shaped free-moving PDMS structure, which can be closed or opened by the pneumatic controls.

Once the magnetic beads are added to a pre-lysis sample, they specifically bind to DNA in the lysate. At this time, the electromagnetic plate placed under the chip is switched to “ON”, which causes the magnetic beads to bind to DNA present in the channel. The electromagnet in the buffer layer 2 plays a role of separating DNA from the original lysis solution. With the increase of the positive pressure and the gradual release of magnetism, DNA adsorbed by the magnetic beads moves forward into the channel. At this time, the magnetic bonding buffer solution in the blister P2 from the buffer layer is released.

The buffer layer 2 further includes a pop-up valve 8 with an eight-pin channel separator. The pop-up blister is filled with 100 μL of washing buffer solution, and 12.5 μL (100/8) of washing buffer solution is added to each channel according to the number of the channels used. The subchannel in the buffer layer 2 is tapered upwards and is connected to the main channel in the feeder layer 1, such that when magnetic bead-bound DNA molecules are present in the main channel of the feeder layer 1, the washing buffer solution from the buffer layer 2 is introduced into the corresponding main channel 5 of the feeder layer 1, to wash the magnetic bead-bound DNAs to remove any debris, waste or other nucleic acids. This step allows the user to obtain high-purity DNA without any contamination.

The buffer layer 2 further includes a pop-up valve 8 with an eight-pin channel separator, which includes three valves for controlling the buffer solution to flow into the channel. The pop-up blister is filled with 100 μL of the elution buffer solution, which can separate the magnetic beads from DNA captured by the magnetic beads, thus enabling the user to obtain pure DNA from the lysate sample at the end of the main channel in the feeder layer 1 outlet of the chip. One to eight collection tubes connected at the outlet allow the user to collect one to eight pure DNA samples from the RapidμFDNA chip.

The pneumatic layer 3 is a pneumatic control layer for controlling the check valve in the buffer layer 2. These pneumatic valves help the user to limit fluid from entering into one subchannel or into all eight subchannels simultaneously. The valve control allows the user to run a single or eight samples for DNA extraction.

The feeder layer 1 is responsible for introducing the original cell lysis solution into a straight channel, the buffer layer 2 is configured to add the magnetic beads, washing and eluting buffer solutions to the lysate sample in sequence, and electromagnetically capture DNA, and the pneumatic layer 3 is configured to provide pneumatic control for the valve in the buffer layer 2. These three layers are made separately, but are connected together and work together, so that the buffer layer 2 and the pneumatic layer 3 eventually flush their materials into the main channel in the feeder layer 1. The overall workflow is shown in FIG. 3.

Materials and devices are as follows.

1. A bottle of 30 ml lysis buffer solution P1 (which is used to separate DNA from non-blood cells, such as fungi, bacteria, or plant cells) and a bottle of 25 ml buffer solution P0 (which is a mixed solution that can lyse blood cells in order to recover DNA).

2. Three pop-up blisters P2-P4 containing buffer solutions.

3. The pop-up blister P2 containing 50 μL of magnetic beads, the pop-up blister P3 containing 100 μL of magnetic binding buffer solution, and the pop-up blister P4 containing 100 μL of elution buffer solution.

4. A microfluidic device with eight sample input ports, a built-in electromagnet and circuit for managing the release of popup contents of the blister at a desired time.

5. A pressure pump for providing positive pressure for all eight sample input ports to push the buffer solution forward in the microfluidic device.

6. Silicone pipe fittings for connecting the pressure pump to the device and eight collection pipes.

For all samples, 1.5 mg of Proteinase K powder is dissolved in 1.5 ml of deionized water to produce a 1 mg/ml Proteinase K solution, which should be sufficient for 75 reactions.

a. Initial Sample 1 (Blood):

200 μl of uncoagulated blood is placed in a 1.5 ml Eppendorf tube.

200 μl of buffer solution is added, vortexed, and incubated at a room temperature for 5 minutes with occasional flipping.

The mixed solution is centrifuged at 5,000 rpm for 10 minutes. 250 μl of supernatant is discarded with a pipette tip without loss of clearly visible white linear cells.

200 μl of buffer solution is added to the tube again, incubated for 5 minutes with occasional flipping, and centrifuged at 5,000 rpm for 10 minutes. The white blood cells debris should settle on the bottom of the tube.

The supernatant is discarded and 20 μl of proteinase K is added to the tube containing cell precipitates.

Uncoagulated blood includes fresh blood, blood stored in EDTA or a test tube containing citrate.

b. Initial Sample 2 (Cultured Cells):

No more than 5×106 cells are centrifuged at 8,000 rpm for at least 10 minutes. The supernatant is discarded, and the cell precipitate is suspended in 20 μl of proteinase K.

c. Initial Sample 3 (Bacteria Cells):

No more than 2×109 cells are centrifuged at 8,000 rpm for at least 10 minutes.

The supernatant is discarded, and the cell precipitate is suspended in 20 μl of proteinase K.

d. Initial Sample 4 (Mammalian and Rodent Tail Tissues):

10 mg of spleen and 20 mg of other mammalian tissues are ground.

It is recommended to use long rat tail sample with a length of 0.6 cm and a mice tail sample with a length of 0.5 cm.

Then two kinds of rodent tissues can be frozen in liquid nitrogen and ground with a pestle and a mortar. Other options for treating the rodent tissues include destroying the tissues by using a homogenizer. All samples should be suspended in 20 μl of proteinase K.

After pretreatment, the samples are incubated at 30° C. for 5 minutes. All samples should be incubated in a refrigerator until DNA extraction.

1. The pretreatment step is followed by a main extraction procedure, which includes the following steps:

1) 10 μg/ml of RNAse A is added into a buffer solution P1, and then is uniformly mixed by vortex (RNAse A is not available in a RapidμFDNA kit).

2) 200 μl of buffer solution P1 (containing RNAse A) is added to a pretreated DNA sample mixture and is mixed for 15 seconds by the pulse vortex to produce a homogeneous solution.

3) The homogeneous solution is inoculated in a 56° C. water bath for 5 minutes.

4) 400 μl of ethanol with a concentration of 50% is added and mixed with a straw or vortex. (Note: 200 μl of ethanol with a concentration of 96% should be added to the blood sample)

5) The sample is transferred to a 1.5 mL Eppendorf tube and is centrifuged at 10,000 rpm for 3 minutes. After centrifugation, only about 500 μL of supernatant is transferred to the RapidμFDNA.

2. Magnetic bonding: (Steps 2 to 5 are fully automated in the RapidμFDNA device).

1) 50 μL of magnetic beads are added to the buffer solution to form a magnetic bonding buffer solution, and 50 μL of magnetic beads in the magnetic bonding buffer are added to the lysate released and pumped into RapidμFDNA via the electronically controlled blister P2.

2) The magnetic beads preferentially bind DNA particles in the lysate.

3) When the lysate is pumped into the RapidμFDNA and magnetic beads is released, the external electromagnet is powered on to attract the bound DNA particles and immobilize the DNA particles to the bottom of the RapidμFDNA.

3. Washing: (Steps 2 to 5 are fully automated in the RapidμFDNA device).

1) As the magnet is gradually powered off and the bound DNA particles are released, the next blister releases the washing buffer solution electronically to remove any pollutants and cell debris from the DNA.

2) The magnet remains powered on throughout the washing process.

3) As the magnet is completely powered off, the liquid is pushed to proceed to the next elution.

4. Elution: (Steps 2 to 5 are fully automated in the RapidμFDNA device).

1) By releasing the elution buffer solution from the next blister, the liquid containing magnetic bead-binding high-purity DNA is separated from the magnetic beads.

2) During elution, the electromagnet is automatically powered off, so that the magnetic beads are separated from the DNA particles.

5. Collection: (Steps 2 to 5 are fully automated in the RapidμFDNA device).

1) At the end of all the first four steps required in the DNA extraction process, the DNA in the sample is collected in a collection tube.

2) The collected DNA can be preserved at −20° C. for up to 3 months.

6. Embedded electronic control system:

1) Each blister includes sufficient reagents for all eight channels. The liquid in each blister can be dispensed into one, two, four or eight channels. This number is the same for various blisters, so three plus four valves are required.

2) Due to the use of c. IT magnet, no ferro (magnetic) material can be placed near the main microfluidic device, which means that the actual extraction of fluid should be completed in advance and then transferred to the main PDMS chip vendor.

3) A pneumatic switch can be used to control the flow of fluid on the main PDMS. The main PDMS includes a pressurized reservoir corresponding to each blister package, and it is known that the flow of actual blister packages is uneven. Prior to use, the main PDMS chip is rinsed to remove any contamination.

4) The time for injecting fluid from a blister package array is controlled by control software. The core of the software is precise timing requirements of the design. However, if there is a mechanical failure, an instruction to stop the process will be received. The release time difference of the buffer solution in each pop-up blister is 30 seconds.

5) The microfluidic device is controlled by a small low-power embedded system controller, such as any low-power ARM embedded system, including Raspberry Pi 4B or Nvidia Jetson. The microfluidic chip is used to move the buffer solution (liquid) to a reservoir and measure the liquid to ensure that the correct amount is transferred.

6) A controller board capable of activating the blister package is connected to the microcontroller, which is used in combination with a motor controller (shielding) board to inject liquid (buffer solution) from the blister package. Because there are four timing points, three control circuits are needed for switching. An L298N H-bridge motor driver or a MosFET driver (e.g., TB6612FNG) may be used.

7) In addition, there are two main valves to be controlled, that is, there are six valves to be switched. In addition, three valve switches are required to select one, two, four or eight test channels on the device. In the case, an IC2 relay board, such as SKU: EP-0099 may be adopted.

8) The microcontroller software also measures and uses the feedback about the successful addition of buffer solution.

9) A supporting power supply is also required.

In accordance with the present disclosure, DNA can be extracted with high throughput from eight samples at the same time without the assistance of an expensive robot. DNA is rapidly separated and extracted, and the time for DNA separation is less than 10 minutes. The device can be repeatedly cleaned for many times. High-quality and high-quantity DNA can be obtained. At present, the test cost for each DNA extraction is about 5-10 dollars, but the cost can be reduced to 1-2 dollars in the present disclosure, which is extremely low.

In the present disclosure, a PDMS microfluidic chip with three layers is provided, all three layers are sandwiched between each other and work as a whole, and the three layers are sealed together.

The release of liquid from a blister ejector and a valve is controlled by the pneumatic controller placed in the pneumatic layer. These pneumatic controllers send electrical pulses to the valve and the blister ejector.

Embodiments of the present specification are described in a progressive manner, each embodiment focuses on the differences from other embodiments, and the same and similar parts between the embodiments may be referred to each other.

Some specific examples are used for illustration of the principles and implementations of the present disclosure. The description of the embodiments is merely used to help illustrate the method of the present disclosure and the core ideas thereof. In addition, those of ordinary skill in the art can make various modifications in terms of specific embodiments and the scope of application in accordance with the teachings of the present disclosure. In conclusion, the content of this specification shall not be construed as a limitation to the present disclosure.

What is claimed is:

1. A microfluidic device for extracting DNA, comprising a feeder layer, a buffer layer and a pneumatic layer which are sealed and stacked in sequence, wherein an electromagnetic plate is arranged between the buffer layer and the pneumatic layer;
the feeder layer is provided with a plurality of main channels and three blister openings, a syringe pump is connected to an inlet of each main channel, and the syringe pump is configured to inject a lysate sample into each main channel;
the buffer layer is provided with segmented buffer channels, each segmented buffer channel is provided with a plurality of subchannels, and each segmented buffer channel corresponds to one pop-up blister; the buffer channel is communicated with the main channel; the pop-up blister is connected to an inlet of each segmented buffer channel; the pop-up blister is arranged at the top of the feeder layer and is communicated with the inlet of each segmented buffer channel after passing through the blister opening, each pop-up blister is filled with different type of buffer solutions, and these buffer solutions comprise a magnetic binding buffer solution, a washing buffer solution and an elution buffer solution;
each segmented buffer channel is provided with a plurality of convergence nodes, a pop-up valve is arranged at each convergence node, and the pop-up valve is pneumatically controlled by the pneumatic layer.

2. The device according to claim 1, wherein the feeder layer, the buffer layer, and the pneumatic layer each have a length of 10 cm and a thickness of 1 mm.

3. The device according to claim 1, wherein a collection device is arranged at an outlet of each main channel, and the collection device is configured to collect a DNA sample.

4. The device according to claim 1, wherein a pop-up blister containing the magnetic binding buffer solution, a pop-up blister containing the washing buffer solution, and a pop-up blister containing the elution buffer solution are arranged in sequence in a direction from the inlet of the main channel to the outlet of the main channel;
the pop-up blister containing the magnetic binding buffer solution, the pop-up blister containing the washing buffer solution and the pop-up blister containing the elution buffer solution are released in sequence in the direction from the inlet of the main channel to the outlet of the main channel to release the magnetic binding buffer solution, the washing buffer solution, and the elution buffer solution in sequence.

5. The device according to claim 1, wherein eight main channels are provided in the feeder layer, and each segmented buffer channel includes eight subchannels in the buffer layer.

6. The device according to claim 5, wherein each segmented buffer channel is provided with an eight-valve channel separator with three pop-up valves.

7. The device according to claim 6, wherein the pop-up valve is a check valve for limiting fluid from entering one subchannel or eight subchannels simultaneously.

8. The microfluidic device for extracting DNA according to claim 7, wherein the pop-up valve is a T-shaped free-moving polydimethylsiloxane structure.

9. The microfluidic device for extracting DNA according to claim 1, wherein the microfluidic device is reusable after washing with a bleaching agent with a concentration of 1% to 2%, and rinsing with double distilled water followed by air-drying.
